(19) United States
(12) Patent Application Publication
(10) Pub. No.: US 2022/0275342 A1
(43) Pub. Date: Sep. 1, 2022

(54) DISSOLVABLE AND DEGRADABLE ARTIFICIAL CIRCULATION SYSTEMS FOR LARGE VOLUME TISSUES

(71) Applicant: BOARD OF REGENTS OF THE UNIVERSITY OF NEBRASKA, Lincoln, NE (US)

(72) Inventors: Yuguo LEI, Lincoln, NE (US); Qiang LI, Cambridge, MA (US); Ou WANG, Lincoln, NE (US)

(21) Appl. No.: 17/625,686

(22) PCT Filed: Jul. 6, 2020
(86) PCT No.: PCT/US2020/040914

§ 371 Date: Jan. 7, 2022

Related U.S. Application Data
(60) Provisional application No. 62/871,825, filed on Jul. 9, 2019.

(51) Int. Cl. C12N 5/071; C12M 3/00; C12M 1/00

(52) U.S. Cl. CPC C12N 5/0691; C12M 21/08; C12M 29/10; C12N 2513/00; C12N 2533/40; C12N 2533/74; C12N 2539/00; C12N 2501/165; C12N 2501/135; C12N 2501/115

(57) ABSTRACT

Embodiments of the disclosure provide a dissolvable or degradable artificial circulation system for engineering, culturing, and integrating large volume tissues. Also provided are methods of using large engineered tissues prepared using the degradable artificial circulation system for clinical applications and for various applications such as large-scale production of therapeutic or consumable products, drug discovery, and toxicity screening.

### CROSS-REFERENCE TO RELATED APPLICATIONS

This application claims priority to U.S. Provisional Application Ser. No. 62/871,825, filed Jul. 9, 2019, which is incorporated herein by reference in its entirety.

### BACKGROUND

Currently, it is still a challenge to make viable pre-vascularized engineered tissues, particularly large tissues having dimensions that exceed the typical diffusion limits of oxygen and nutrients into an artificial tissue. Available methods employ polymer hollow fibers as an artificial circulation system. Since polymer fibers are neither degradable nor dissolvable, the “vessels” of such artificial circulation systems must be removed prior to implantation, which damages the tissue. Polymer tubes also have poor diffusion properties, meaning it is difficult to obtain adequate dispersal of oxygen and nutrients throughout large engineered tissues to sustain their viability in vitro and upon transplantation. Accordingly, there remains an unmet need in the art for a robust artificial circulation system for scalable, cost-effective preparation and transplantation of large engineered tissues without a need to retrieve material prior to or following transplantation.

### BRIEF SUMMARY OF THE DISCLOSURE

In a first aspect, provided herein is a method for preparing a large engineered tissue. In some cases, the method comprises or consists essentially of the following steps: (a) seeding cells onto a three-dimensional scaffold comprising one or more hollow biomaterial tubes, each tube comprising a first tube end and a second tube end; (b) circulating a culture medium through the hollow biomaterial tubes, wherein circulating comprising forming a fluid circuit between the one or more hollow biomaterial tubes and a directional fluid pumping device comprising a first inlet, a second inlet, and a reservoir, wherein the first tube end is in fluid contact with the first inlet and the second tube end is in fluid contact with the second inlet, and wherein the first and second inlets introduce the culture medium from the reservoir into one or more hollow biomaterial tubes; and (c) culturing the seeded scaffold under conditions that promote one or more of proliferation, differentiation, and maturation of the seeded cells to form an engineered tissue having a thickness greater than 1 mm in at least one dimension. The scaffold can comprise a plurality of hollow biomaterial tubes, each hollow biomaterial tube spaced apart to support efficient nutrient diffusion throughout the whole engineered tissue. The cells can be cell spheroids and seeding can comprise placing the cell spheroids between hollow biomaterial tubes of the scaffold. Seeding can comprise placing single cells adjacent to an outer surface of the one or more biomaterial tubes. The outer surface of the one or more biomaterial tubes can comprise cell adhesion molecules. The biomaterial tubes can comprise a hydrogel. The hydrogel can be degradable. The hydrogel can be dissolvable. The biomaterial tubes can comprise alginate. The alginate can comprise alginate acid polymers, sodium alginate polymers, or modified alginate polymers, or combinations thereof. The alginate can be dissolvable. The 3D scaffold can be seeded with cells. The cells can be selected from embryonic stem cells, induced pluripotent stem cells, cells differentiated from embryonic stem cells or induced pluripotent stem cells, cells reprogrammed from other cell types, primary cells, endothelial cells, umbilical vein endothelial cells, vascular smooth muscle cells, cancer cells, T cells, tissue stem cells, mammalian cells, plant cells, yeast and bacterial cells, or a combination thereof. In some cases, the method further comprises seeding the hollow biomaterial tubes with cells. The cells can comprise endothelial cells, vascular smooth muscle cells, or a combination thereof. The hollow biomaterial tubes can be further seeded with growth factors. In some cases, the engineered tissue comprises blood vessels.

In another aspect, provided herein is an artificial tissue circulation system. The system can comprise or consist essentially of (a) a directional fluid pumping device having a first inlet, a second inlet, and a reservoir; and (b) a three-dimensional (3D) biocompatible scaffold comprising one or more hollow biomaterial tubes, each tube comprising a first end and a second end, wherein the first end is in fluid contact with the first inlet and the second end is in fluid contact with the second inlet, and wherein the first and second inlets are operable for introducing a fluid from the reservoir into the hollow biomaterial tube, thereby forming a fluid circuit between the directional fluid pumping device and the hollow biomaterial tube. The scaffold can comprise a plurality of hollow biomaterial tubes. The hollow biomaterial tube can comprise a hydrogel. The hydrogel can comprise alginate. The alginate can comprise alginate acid polymers, sodium alginate polymers, or modified alginate polymers, or combinations thereof. The 3D scaffold can be seeded with cells. The cells can be selected from embryonic stem cells, induced pluripotent stem cells, cells differentiated from embryonic stem cells or induced pluripotent stem cells, cells reprogrammed from other cell types, primary cells, endothelial cells, umbilical vein endothelial cells, vascular smooth muscle cells, cancer cells, T cells, tissue stem cells, mammalian cells, plant cells, yeast and bacterial cells, or a combination thereof. The reservoir can comprise a cell culture medium.

These and other advantages and features of the invention will become more apparent from the following detailed description of the preferred embodiments of the invention when viewed in conjunction with the accompanying drawings.

### BRIEF DESCRIPTION OF THE DRAWINGS

FIG. 1 is an illustration depicting a degradable artificial circulation system of this disclosure. A large engineered tissue is prepared by culturing cells in a three-dimensional (3D) scaffold that comprises a plurality of hollow biomaterial tubes. A perfusion system circulates a cell culture medium through the hollow biomaterial tubes. Nutrients, oxygen and growth factors pass through the tubes to feed cells in the 3D tissue, and metabolic wastes are collected to the tube and carried away during the in vitro culture and after transplantation. These tubes can be dissolved or degraded with bio-compatible reagents or by the body in vitro or in vivo. Multiple hydrogel tubes can be used in one tissue. Multi-direction circulations can be achieved with multiple sets of hydrogel tubes

FIG. 2 illustrates a configuration comprising multiple, multi-directional hollow biomaterial tubes that mimic an artificial blood-lymphatic vessel system. In this configuration, two sets of hollow biomaterial tubes are used. The red tubes serve as blood vessels for flowing in the fresh cell culture medium. The blue tubes serve as a lymph vessel system to collect the exhausted medium. A perfusion system is used to circulate fresh culture medium through the red tubes for diffusion into the 3D scaffold. Exhausted medium diffuses into the blue tubes and is removed from the tissue.

FIG. 3 illustrates host integration of a large engineered tissue comprising multiple, multi-directional hollow biomaterial tubes. The blue set of tubes are dissolved before tissue transplantation. Optionally, vascular lineage cells such as endothelial cells (ECs) and vascular smooth muscle cells (VSMCs) are cultured within the blue tubes to form blood vessels prior to transplantation. Following transplantation, a perfusion system is used to flow a culture medium comprising growth factors such as VEGF and PDGF through the red tubes. Attracted by growth factors and nutrients, host blood vessels grow into channels created by the dissolved blue hydrogel tubes. The medium flew through the red tubes will be gradually reduced when the host circulation is gradually established in the tissue. Once the host circulation is completely established, the external medium perfusion is stopped. The red hydrogel tubes are gradually absorbed by the body.

FIGS. 4A-4B present images of a perfusion system to test one-directional flow. A low-cost perfusion pump is used to drive the liquid flow. Alginate hydrogel tubes (outer diameter—500 μm, inner diameter—400 μm, 2% alginate) were embedded in an agarose hydrogel (3%) with 0.5 cm thickness. Blue dye is perfused through hydrogel tubes unidirectionally and diffuses quickly into a 3D hydrogel matrix.

FIGS. 5A-5E demonstrate diffusion of a large biomolecule, here FITC-dextran, in a degradable artificial circulation system. The configuration is the same as the one described in FIG. 4. 0.5% FITC labelled dextran was perfused. FIG. 5A shows fluorescent images of FITC-dextran-20K and FITC-dextran-40K from 1 minute to 90 minutes. FIGS. 5B, C quantify the fluorescent intensities of FITC-dextran-20K and FITC-dextran-40K from 1 minute to 90 minutes cross the 3D tissue. FIGS. 5D, E show the diffusion rate of FITC-dextran-20K and FITC-dextran-40K. The results show large biomolecules can quickly pass through the hydrogel tubes and diffuse in the 3D tissue.

FIG. 6 presents images of a bioreactor and hollow alginate tubes for preparing large engineered tissues. In this embodiment, the bioreactor is a sandwich structure comprising a top cover, bottom cover, and chamber block. A 3D tissue can be prepared and matured in the bioreactor.

FIGS. 7A-7E demonstrates engineering a large tissue using an artificial circulation system. FIG. 7A shows a hollow alginate tube. FIG. 7B shows aligned hollow alginate tubes in a bioreactor chamber. FIG. 7C shows human pluripotent stem cells (hPSCs) suspended in a hydrogel precursor solution (0.8% agarose solution). FIG. 7D shows a hPSC-seeded tissue (width: 1 cm; length: 1 cm; thickness: 0.5 cm) in the bioreactor. FIG. 7E shows the cell spheroids and alginate tubes within the large tissue.

FIGS. 8A-8D demonstrate a large engineered tissue culture of human pluripotent stem cells (hPSCs) in a degradable artificial circulation system of this disclosure. FIG. 8A shows an overview of a bioreactor for culturing large tissues, which includes an oxygen-permeable plastic bag for culture medium, a pump for medium perfusion, and a bioreactor chamber for culturing the large tissue. FIG. 8B demonstrates live cells in the large tissue on culture day 1 with medium perfusion (green: live cells; red: dead cells). FIG. 8C shows live cells in the large tissue on culture day 3 with medium perfusion (green: live cells; red: dead cells). FIG. 8C shows that cells in the large tissue on culture day 3 without medium perfusion are dead (green: live cells; red: dead cells). These results demonstrate that the artificial circulation system described herein is sufficient to support live cells for multiple days.

FIG. 9 demonstrates a large engineered tissue culture of D1 mesenchymal stem cells in a degradable artificial circulation system of this disclosure. All cells are alive (green) after 1 and 6 days of culture with medium perfusion. These results demonstrate that the artificial circulation system described herein is supports longer culture of large engineered tissues in a bioreactor.

### DETAILED DESCRIPTION

The systems and methods provided herein are based at least in part on the inventor's development of dissolvable and/or degradable artificial circulation systems useful for engineering and integrating large volume tissues. In conventional systems, an engineered tissue is made and cultured in a bioreactor in order for the tissue to mature prior to transplantation or implantation in a host body. When transplantation is successful, the transplanted/implanted tissue survives, integrates with the host tissue, and becomes functional. Large tissues face two critical mass transport problems prior to and after transplantation. The diffusion limit for nutrients and oxygen in an artificial tissue is typically less than 500 For tissues larger than 500 μm, the tissue needs at least one blood vessel or capillary within that diffusion limit to supply blood, nutrients, and oxygen throughout the tissue. Since there is no available technology to build functional blood vessels into an engineered tissue, large engineered tissues (e.g., >1 mm thick up to thicknesses of several centimeters) will die during the in vitro culture period and/or after transplantation. As described herein, the inventor developed an artificial circulation system that makes it possible to sustain living engineered large tissues for extended culturing periods.

Advantages of the systems and methods provided herein are multifold. For example, the biocompatible and biodegradable tubes are easily modified, scalable between from microscale to macroscale, and do not require removal after transplantation. Unlike conventional hydrogel-based technologies, the methods and compositions of this disclosure are suited for large tissue engineering. In particular, the methods and compositions of this disclosure advantageously comprise tubes that can be dissolved or degraded with EDTA, or enzymes, or by the body without retrieval and can employ multiple tubes in a single tissue to achieve multi-directional circulation. As demonstrated herein, an artificial tissue comprising multiple, multi-directional tubes can recapitulate the blood-lymph system.

Accordingly, in a first aspect, provided herein is an artificial circulation system for producing a large engineered tissue. As used herein, “artificial circulation system” refers to methods by which fluids, such as a nutrient-containing culture medium or blood, flow in a controlled way through an engineered tissue or organ. As described herein, artificial circulation systems recapitulate, outside of the body, the nutrient delivery and gas exchange systems provided by the vascular system, heart, and lungs, thus simulating the circulatory system. Preferably, the artificial circulation system is dissolvable and/or degradable. As used herein, the term “dissolvable” refers to a biomaterial's capacity to be dissolved by a reagent. As used herein, the term “degradable” refers to a biomaterial's capacity to be broken down into portions or pieces of the biomaterial polymers.

In some embodiments, the artificial circulation system comprises or consists essentially of (a) a directional fluid pumping device having an inlet port, an outlet port, and a fluid reservoir; and (b) a three-dimensional (3D) scaffold/tissue comprising one or more hollow biomaterial tubes or channels, each comprising a first end and a second end, the first end connecting the directional fluid pumping device outlet port and the second end connecting the directional fluid pumping device inlet port to form a fluid circuit.

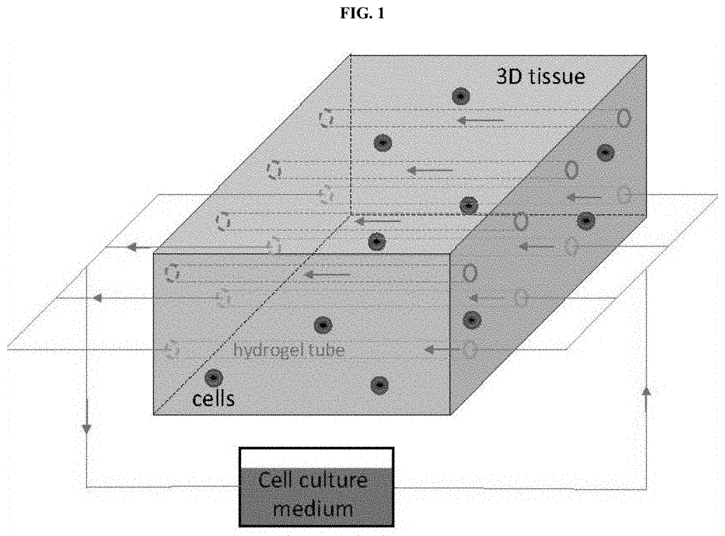

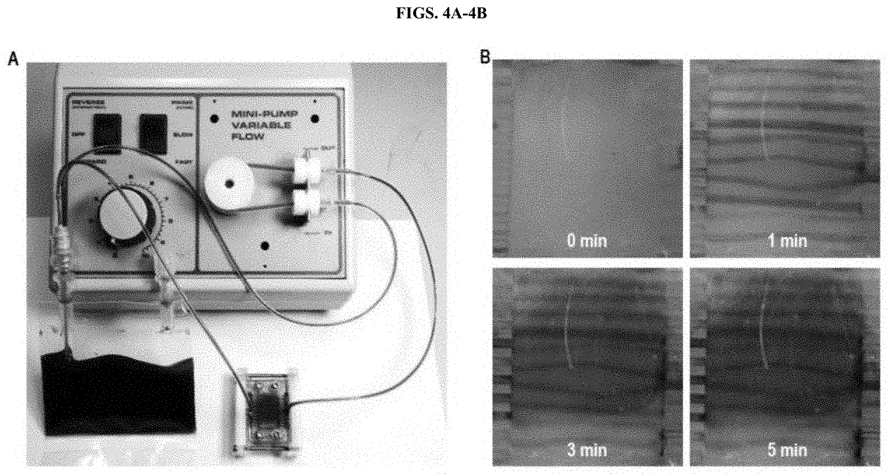

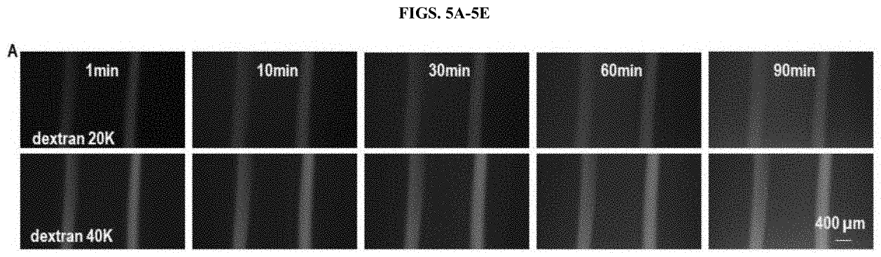

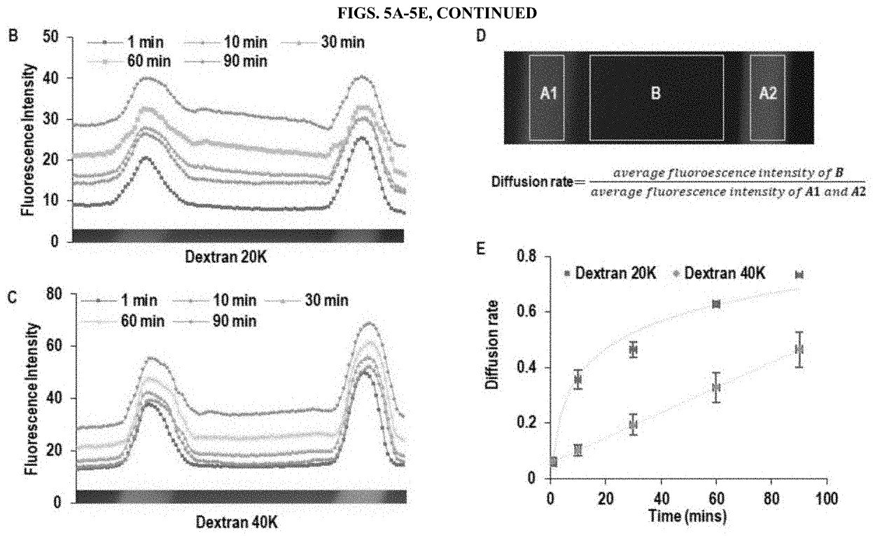

Referring to FIG. 1, fluid (e.g., culture medium) is circulated from a fluid reservoir through the one or more hollow biomaterial tubes located within a 3D scaffold/tissue. In some cases, fluid flow is unidirectional, where fluid circulates through the hollow biomaterial tubes in a single direction. In some cases, hollow biomaterial tubes are substantially parallel to each other (as depicted in FIG. 1), but it will be appreciated that the hollow tubes need not be parallel or substantially parallel to each other. Other configurations are suitable. In some cases, the ends of each tube are located on opposing sides of the 3D scaffold/tissue. In some cases, the a first end of a hollow biomaterial tube is connected to a first inlet port of a directional fluid pumping device, and a second end of the tube is connected to a second inlet port of a directional fluid pumping device such that, when the pumping device is operational, fluid from the reservoir is pumped into the biocompatible scaffold via the fluid transport tube. Unidirectional flow is also demonstrated in FIGS. 4A-4B and FIG. 5A.

Referring to FIG. 2, it may be advantageous in some cases for the 3D scaffold to comprise multiple hollow biomaterial tubes or sets of tubes. As depicted in FIG. 2, the hollow biomaterial tubes can be closed on one end such that fluid flows into the tube and diffuse through the scaffold material. A second set of hollow biomaterial tubes, each tube of this set also having one closed end, can be connected to the artificial circulation system to pump exhausted culture medium from the scaffold. In such a system, fluid flow is unidirectional but involves multiple sets of hollow biomaterial tubes. The hollow tube configuration illustrated in FIG. 2 recapitulates the blood-lymphatic vessel system of the human body.

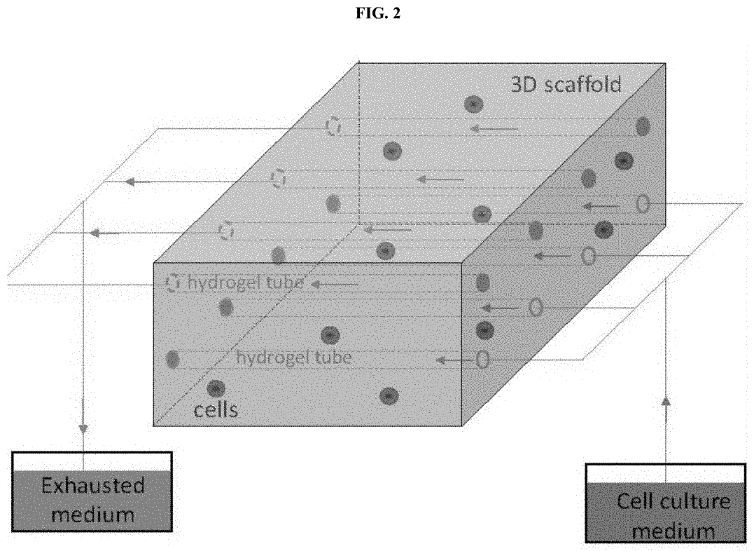

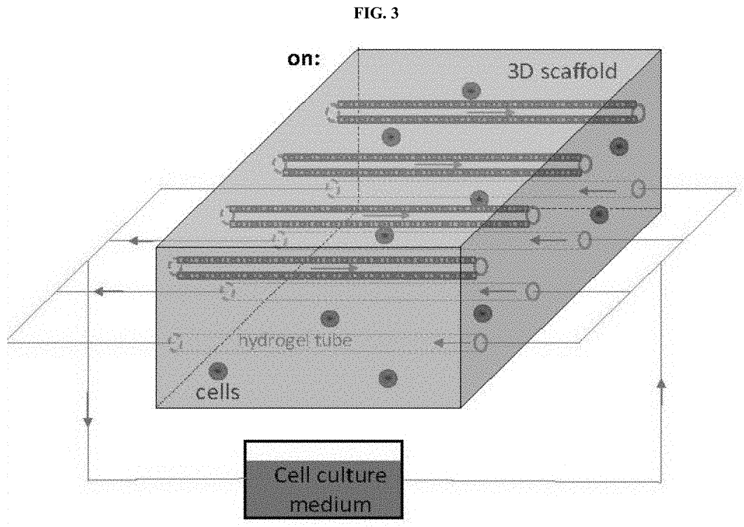

As illustrated in FIG. 3, the system can comprise multiple, multi-directional hollow biomaterial tubes. Following culture of the scaffold/tissue to obtain the large engineered tissue, a portion of the hollow biomaterial tubes can be dissolved or degraded prior to transplantation. In other cases, a portion of the hollow biomaterial tubes can be seeded with vascular lineage cells such as endothelial cells (ECs) and vascular smooth muscle cells (VSMCs), with or without growth factors (e.g., a VEGF, a PDGF), to initial vessel formation prior to transplantation. Without being bound to any particular theory or mechanism, it is believe that host blood vessels, attracted by the presence of vascular lineage cells and growth factors, will grow into channels created by the dissolved blue hydrogel tubes. The medium flowing through the red tubes can be gradually reduced as host circulation is gradually established in the tissue. Once the host circulation is fully established, the external medium perfusion is stopped. The red hydrogel tubes are gradually adsorbed by the body.

In another aspect, provided herein is a method for preparing a large engineered tissue. The method can comprise or consist essentially of (a) seeding cells onto a three-dimensional scaffold comprising one or more hollow biomaterial tubes, each tubes comprising a first tube end and a second tube end; alternatively, single cells or cell spheroids can be directly seeded to the inter-tube space without using scaffold; (b) circulating a culture medium through the hollow biomaterial tubes. Preferably, circulating comprising forming a fluid circuit between the one or more hollow biomaterial tubes and a directional fluid pumping device comprising a first inlet, a second inlet, and a reservoir, where the first tube end is in fluid contact with the first inlet and the second tube end is in fluid contact with the second inlet, and where the first and second inlets introduce the culture medium from the reservoir into one or more hollow biomaterial tubes. With artificial circulation of the culture medium, the seeded scaffold is cultured under conditions that promote survival, proliferation, and differentiation of the seeded cells to form an engineered tissue having a thickness greater than 1 mm (>1000 μm) in at least one dimension. Preferably, the engineered tissue has a thickness greater than 1 mm in all three dimensions.

In some embodiments, cells are cultured in or on the 3D scaffold to make a large, three-dimensional engineered tissue using the artificial circulation system. As used herein, the terms “large” or “large-scale” refers to a three-dimensional tissue product having a thickness of at least 1 mm in at least one dimension. Preferably, three-dimensional tissue product having a thickness of at least 1 mm in all three dimensions. In some cases, a large engineered tissue has a thickness of at least 1 mm in at least two dimensions and least 1 cm in at least one dimension. As described herein, most cells within the body are found no more than 100-200 μm from the nearest capillary, with this spacing providing sufficient diffusion of oxygen, nutrients, and waste products to support and maintain viable tissue. Likewise, when tissues grown in the laboratory using conventional methods and are implanted into the body, this diffusion limitation allows only cells within 100-200 μm from the nearest capillary to survive. The systems and methods provided herein, however, advantageously provide viable engineered tissues having much greater thicknesses. Preferably, the 3D scaffold comprises hollow biomaterial tubes or channels spaced apart such that the resulting engineered tissue comprises a simulated circulatory system through which nutrients, oxygen can diffuse throughout the entire tissue and, in some embodiments, waste products can be removed from the tissue. Where the engineered tissue has only one hollow biomaterial tube, the tube should be located centrally and the overall tissue size should be about 1 mm (about 1000 μm).

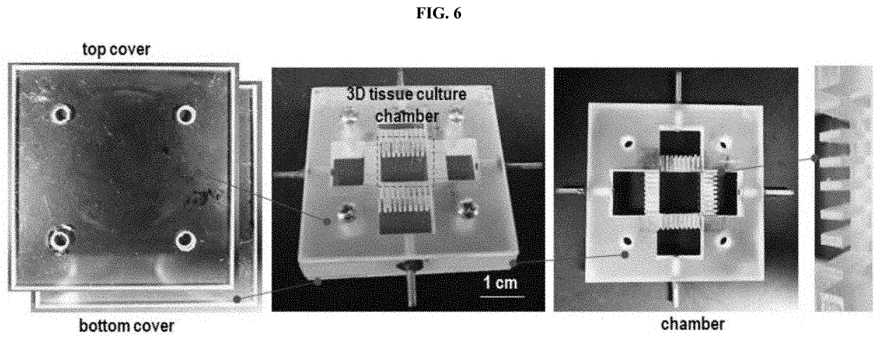

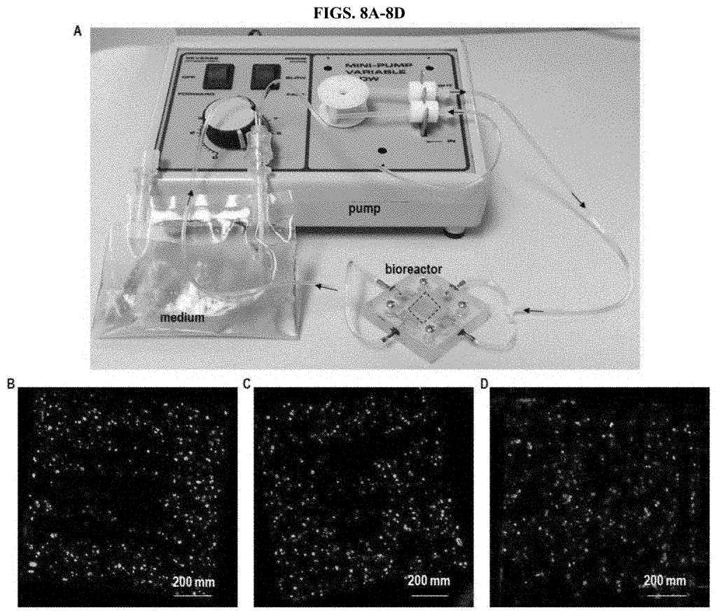

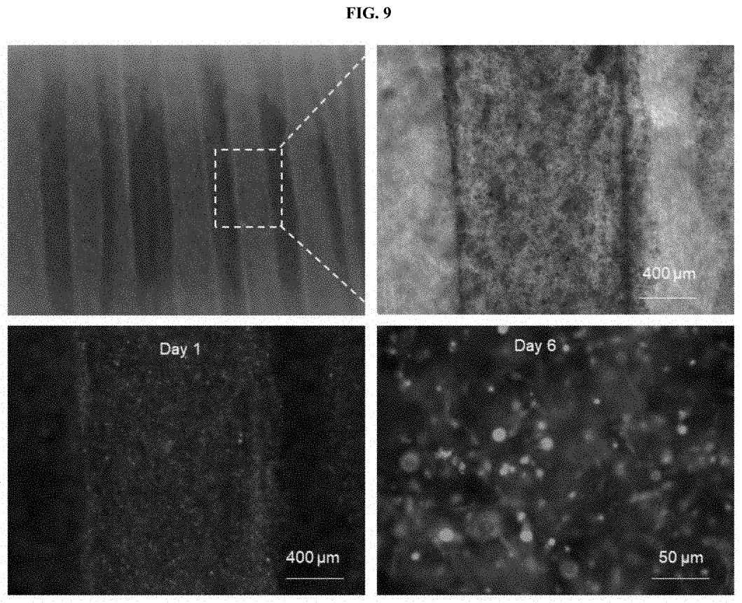

When the artificial circulatory system is active, fluid (e.g., a cell culture medium) is pumped through the hollow biomaterial tubes, providing nutrients and oxygen into engineered tissues forming in or around the 3D scaffold. Preferably, the tubes are formed from a porous material such as a hydrogel, allowing nutrients, oxygen, and metabolic waste to diffuse into and out of the hollow tubes. The hollow tubes can be arranged in any useful configuration. By way of example, FIGS. 4 and 5 illustrate tubes configured in a single direction relative to the 3D scaffold/tissue. Alternative configurations, such as those shown in FIGS. 6-9, can be used for fluid flow in multiple directions. Multiple hollow biomaterial tubes can be used in one tissue, and multi-direction circulation can be achieved with multiple sets of hollow biomaterial tubes. Depending on the overall size of the desired engineered tissue, it may be advantageous to provide a high density of tubes within the 3D scaffold/tissue for medium circulation during in vitro culture and serve as a degradable artificial capillary bed after transplantation.

Any appropriate biocompatible material can be used for the hollow biomaterials tubes including, for example, hydrogel. As used herein, the term “hydrogel” refers to a highly hydrated porous material comprising synthetic or biological components formed when an organic polymer (natural or synthetic) is cross-linked via covalent, ionic, or hydrogen bonds to create an open-lattice structure that entraps water molecules to form a gel. Hydrogels appropriate for engineering large artificial tissues include, without limitation, synthetic hydrogels, bioactive hydrogels, biocompatible hydrogels, cytocompatible hydrogels, chemically defined hydrogels, chemically-defined synthetic hydrogels, and proteolytically degradable hydrogels. Preferably, hydrogels used for the engineered tissues described herein are biocompatible. As used herein, the term “biocompatible” refers to the ability of a material (e.g., hydrogel) to perform as a substrate that will support cellular activity, including the facilitation of molecular and mechanical signaling systems, in order to permit proper cell self-assembly or cellular function such as tissue formation, production of soluble bioactive molecules (e.g., growth factors), or specific cell behaviors such as migration and proliferation. In some cases, “biocompatibility” means the absence of components having cell- or tissue-damaging effects. As used herein, the term “cytocompatible” means the hydrogel material is substantially non-cytotoxic and produces no, or essentially no, cytotoxic degradation products.

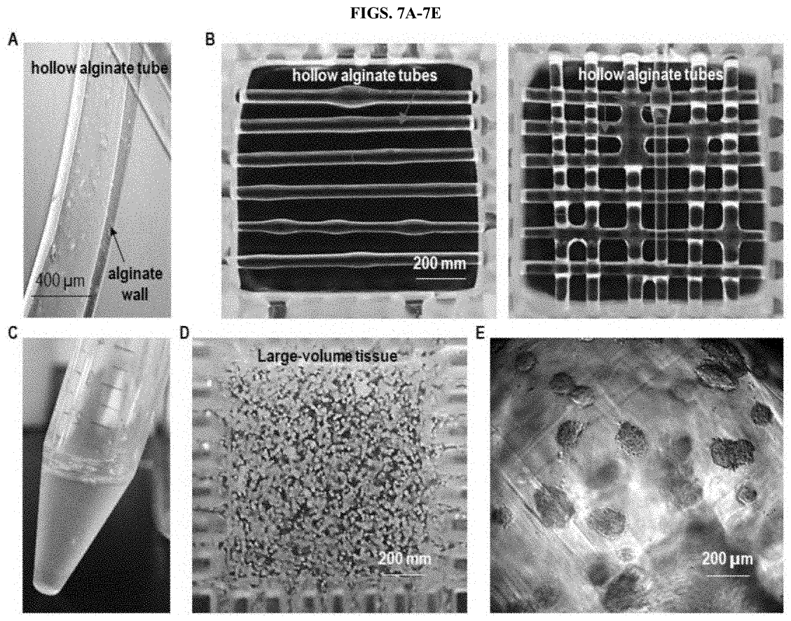

In preferred embodiments, the hollow biocompatible tubes comprise hydrogel. Any appropriate method can be used to prepare hollow hydrogel tubes. In some cases, hollow hydrogel tubes are prepared according to the methods described by the inventor in U.S. Patent Publication 2018/0327703, which is incorporated herein as if set forth in its entirety. In some cases, the hollow biocompatible tubes have a diameter of about 400 μm. See FIG. 7A. Dimensions of the hollow tubes can be scaled up or down based on the particular application, tissue type, or tissue size.

In some embodiments, the hydrogel comprises polyethylene glycol (PEG), polyvinyl alcohol (PVA), polyvinyl pyrrolidone (PVP), polyacrylamides, or polysaccharides, or a combination thereof. Exemplary polysaccharide hydrogels are made by crosslinking natural or semi-synthetic polysaccharides such as alginate, carboxymethylcellulose, hyaluronic acid, and chitosan. Alginate hydrogels can comprise alginate acid polymers, sodium alginate polymers, modified alginate polymers, or combinations thereof.

In preferred embodiments, hollow biocompatible tubes are dissolvable or degradable with varied dissolution or degradation rates and/or dissolution or degradation mechanisms. For example, the hollow biocompatible tubes can comprise alginate hydrogel which is chemically dissolvable using a chemical dissolvent such as ethylene diamine tetraacetic acid (EDTA), ethylene glycol tetraacetic acid (EGTA), and an alginate lyase solution. In some cases, hollow biocompatible tubes comprises hydrogel material crosslinked with one or more peptides sensitive to protease, such that the hollow biocompatible tubes are proteolytically degradable.

Any materials suitable for cell growth and tissue engineering can be used as a scaffold. As used herein, the term “scaffold” refers to a substrate that supports cell growth, proliferation, differentiation, and/or maturation to form a three-dimensional tissue. Generally, scaffolds are three-dimensional substrates fabricated, at least in part, from synthetic or natural polymers or a mixture/composite thereof. Scaffold materials should be non-toxic, cytocompatible, and biocompatible to support cell growth and to minimize inflammation after transplantation. Preferably, scaffold materials are completely degraded in vivo with time, once cells of the implanted tissue have been guided to perform full functions and roles as tissues. Examples of commercially available synthetic biodegradable polymers include, without limitation, polyglycolic acid (PGA), polylactic acid (PLA), polylactic acid-glycolic acid copolymer (PLGA), poly-ε-caprolactone (PCL), and derivatives and copolymers thereof. Examples of natural biodegradable polymers used as scaffold materials include, without limitation, collagen, alginate, hyaluronic acid, gelatin, chitosan, and fibrin. The scaffold may be in various forms, such as sponges, gels, fibers, microbeads, or injectable hydrogels.

In some cases, cell spheroids can be directly placed between tubes (e.g., hydrogel tubes). Spheroids have their own extracellular matrix and can fuse to form a tissue. In some cases, single cells can be seeded and adhere to the outer surface of the tubes. In some cases, the tubes are modified to comprise cell adhesion molecules to which the single cells can adhere.

As used herein, the terms “synthetic” and “engineered” are used interchangeably and refer to a non-naturally occurring tissue material that has been created or modified by the hand of man (e.g., prepared in vitro using natural or synthetic materials) or is derived using such material. In some cases, cells used to produce the engineered tissue material are wild-type cells or may contain one or more synthetic or genetically engineered nucleic acids (e.g., a nucleic acid containing at least one artificially created insertion, deletion, inversion, or substitution relative to the sequence found in its naturally occurring counterpart). Cells comprising one or more synthetic or engineered nucleic acids are considered to be an engineered cell. As used herein, the term “bioengineered” generally refers to a tissue prepared in vitro using biological techniques including, for example, techniques of cell biology, biochemistry, tissue culture, and materials science. As used herein, the term “tissue” refers to aggregates of cells. As used herein, the term “engineered tissue” (or similar term) refers to a tissue prepared in accordance with the systems and methods of this disclosure. Preferably, an engineered tissue displays physical characteristics typical of the type of the tissue in vivo and functional characteristics typical of the type of the tissue in vivo, i.e., has a functional activity.

In some embodiments, the 3D scaffold/tissue is seeded with cells to prepare a large engineered tissue. Exemplary cell types appropriate for use in connection with the artificial circulation systems of this disclosure include, without limitation, embryonic stem cells; induced pluripotent stem cells, naive pluripotent stem cells; cell aggregates; cell spheroids; embryoid bodies or organoids comprising embryonic stem cells and/or induced pluripotent stem cells; cells differentiated from embryonic stem cells, induced pluripotent stem cells, and naive pluripotent stem cells; cells reprogrammed from other cell types (e.g., cells reprogrammed from human fibroblasts); primary cells; endothelial cells, smooth muscle cells, fibroblasts, human umbilical vein endothelial cells; cancer cells; immune cells; tissue stem cells (e.g., mesenchymal stem cells); cell lines; plant cells; yeast, and bacterial cells, or combinations thereof. Although human cells are preferred for the systems and methods of this disclosure, it may be advantageous in some instances to prepare large engineered tissues comprising non-human cells. For example, it may be advantageous to use cells obtained from other mammalian species including, without limitation, equine, canine, porcine, bovine, feline, caprine, murine, and ovine species. Cell donors may vary in development and age. In some cases, the scaffold is seeded with a single cell type (e.g., SMCs, endothelial cells). In other cases, the scaffold is seeded with multiple cell types (e.g., fibroblasts and human umbilical vein endothelial cells).

Preferably, seeded cells of the 3D scaffold/tissue will undergo normal biological processes of cell growth, proliferation, differentiation, and/or maturation to form a three-dimensional tissue.

In some embodiments, the engineered tissue comprises one or more populations of cells derived from the same subject into which the engineered tissue to be implanted (i.e., autologous cells). In some of the above embodiments, the engineered tissue comprises one or more populations of cells derived from stem cells or progenitor cells, such as human pluripotent stem cells (e.g., human embryonic stem cells, human induced pluripotent stem cells).

It may be advantageous for some applications for the engineered tissue to be a cell-free scaffold into which nutrients are perfused using an artificial circulation system described herein. Such cell free scaffolds can be implanted to attract host cells, thereby forming a tissue.

In some embodiments, the 3D scaffold is seeded with recombinant or genetically-modified cells in place of or in addition to unmodified or wild-type (“normal”) cells. For example, it can be advantageous in some cases to include recombinant/genetically-modified cells that produce recombinant cell products, growth factors, hormones, peptides or proteins (e.g., detectable reporter proteins) for a continuous amount of time or as needed such as, for example, when biologically, chemically, or thermally signaled due to the conditions present in culture. Procedures for producing genetically modified cells are generally known in the art, and are described in Sambrook et al, Molecular Cloning, A Laboratory Manual, Cold Spring Harbor Press, Cold Spring Harbor, N.Y. (1989), incorporated herein by reference.

Cell culture conditions, including the type of culture medium used with the system, will vary depending on the type of and cell(s) the number of cells seeded into the scaffold, the type of and cell(s) the number of cells introduced into hollow biomaterial tubes, the desired engineered tissue, and the size of the scaffold or engineered tissue. For example, when the seeded cells are human pluripotent stem cells, suitable culture media include, but are not limited to, mTeSR®, E8™, Essential 8 (Thermal Fisher/Life Technologies Inc.), and TeSR-E8 (Stem Cell Technologies) media.

In some embodiments, the cell culture medium is supplemented with angiogenic factors such as a vascular endothelial growth factor (VEGF), basic fibroblast growth factor (bFGF), and platelet-derived growth factor (PDGF), among others. Circulating such vascular growth factors through an engineered tissue using the artificial circulation system provided herein is preferable to coating or loading scaffold material with pro-angiogenic factors because of their short half-lives, high rates of degradation, uneven distribution, and rapid diffusion throughout the material. In addition, the artificial circulation system provides control over the timing and location of vascular growth factor delivery. In some cases, a culture medium supplemented with one or more angiogenic factors such as VEGF and PDGF is circulated through the hollow biomaterial tubes after transplantation in order to promote the gradual establishment of host circulation within the large engineered tissue, providing a seamless transition. In this manner, hydrogel tubes serve as artificial capillaries after transplantation until such time that host vascular cells migrate and establish vessels within the large engineered tissue.

Large engineered tissues prepared according to the systems and methods provided herein can be useful for various in vitro and in vivo applications. In some examples, a large engineered tissue can be used for tissue transplantation, as tissue implants, and for regenerative therapies. In another example, large engineered tissues prepared according to the systems and methods provided herein are used for in vitro drug discovery and drug testing (e.g., toxicity testing).

In another example, large engineered tissues prepared according to the systems and methods provided herein provide a source of in vitro cultured comestible products such as non-human cultured meat products. As used herein, the term “comestible” means suitable and adapted to be eaten by a human being or a non-human animal. For example, a 3D scaffold/tissue of this disclosure can be seeded with a plurality of cells, including progenitor cell types, selected to approximate those found in traditional meat products. In some cases, a 3D scaffold/tissue of this disclosure can be seeded with a plurality of non-human cells comprising one or more of myocytes, fibroblasts, adipose cells, epithelial cells, connective tissue, multipotent cell types, and undifferentiated cells.

In a further example, large engineered tissues prepared according to the systems and methods provided herein can be used for in vitro production of recombinant proteins. For instance, cells seeded into the 3D scaffold/tissue can be genetically engineered to comprise one or more nucleic acid sequences encoding a recombinant protein of interest. Expression of the nucleic acid sequences in such cells of the 3D scaffold/tissue may yield production of that protein. In this manner, large engineered tissues of this disclosure are useful for large-scale protein production. In some cases, the nucleic acid that encodes a therapeutically or commercially important recombinant protein. In some cases, the recombinant proteins are recombinant therapeutic proteins. As used herein, the term “therapeutic protein” refers to a protein or portion thereof (e.g., protein fragment) that has been sufficiently purified or isolated from contaminating proteins, lipids, and nucleic acids (e.g., contaminating proteins, lipids, and nucleic acids present in a liquid culture medium or from a host cell and biological contaminants (e.g., viral and bacterial contaminants), and can be formulated into a pharmaceutical agent without any further substantial purification and/or decontamination step. Therapeutic proteins that can be produced according to these methods include, without limitation, microbicides, immunoglobulins, vaccines, immunogenic or antigenic proteins or protein fragments, antigens, growth factors, growth hormones, cytokines, insulin, erythropoietin, clotting factors, regulatory proteins, structural proteins, transport proteins, transcription factors, antibodies, enzymes, and ribozymes. Any appropriate transformation systems, including stable transformation and transient expression, can be used to introduce nucleic acids into cells that have been or will be seeded into the 3D scaffold/tissue. In some cases, a method of this disclosure further comprises isolating and purifying the recombinant protein. In some cases, the recombinant protein is a secreted protein, meaning that the protein originally contained at least one secretion signal sequence when it is translated within a mammalian cell, and through, at least in part, enzymatic cleavage of the secretion signal sequence in the mammalian cell, is secreted at least partially into the extracellular space (e.g., a liquid culture medium). Skilled practitioners will appreciate that a “secreted” protein need not dissociate entirely from the cell to be considered a secreted protein.

Engineered tissues for use in clinical applications must be obtained in accordance with regulations imposed by governmental agencies such as the U.S. Food and Drug Administration. Accordingly, in exemplary embodiments, the methods provided herein are conducted in accordance with Good Manufacturing Practices (GMPs), Good Tissue Practices (GTPs), and Good Laboratory Practices (GLPs). Reagents comprising animal derived components are not used, and all reagents are purchased from sources that are GMP-compliant. In the context of preparing large engineered tissues for use as a transplant tissue or an implant in humans, GTPs govern cell donor consent, traceability, and infectious disease screening, whereas GMPs are relevant to the facility, processes, testing, and practices to produce consistently safe and effective products for human use. See Lu et al. Stem Cells 27: 2126-2135 (2009). Where appropriate, oversight of patient protocols by agencies and institutional panels is envisioned to ensure that informed consent is obtained; safety, bioactivity, appropriate dosage, and efficacy of products are studied in phases; results are statistically significant; and ethical guidelines are followed.

This disclosure is presented to enable a person skilled in the art to make and use embodiments described herein. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

It is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting.

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Values expressed in a range format should be interpreted in a manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt % to about 5 wt %, but also the individual concentrations (e.g., 1%, 2%, 3%, and 4%) and the sub-ranges (e.g., 0.1% to 0.5%, 1.1% to 2.2%, and 3.3% to 4.4%) within the indicated range.

The invention has been described according to one or more preferred embodiments, and it should be appreciated that many equivalents, alternatives, variations, and modifications, aside from those expressly stated, are possible and within the scope of the invention.

We claim:

1. A method for preparing a large engineered tissue, the method comprising
(a) seeding cells onto a three-dimensional scaffold comprising one or more hollow biomaterial tubes, each tube comprising a first tube end and a second tube end;
(b) circulating a culture medium through the hollow biomaterial tubes, wherein circulating comprising forming a fluid circuit between the one or more hollow biomaterial tubes and a directional fluid pumping device comprising a first inlet, a second inlet, and a reservoir, wherein the first tube end is in fluid contact with the first inlet and the second tube end is in fluid contact with the second inlet, and wherein the first and second inlets introduce the culture medium from the reservoir into one or more hollow biomaterial tubes; and
(c) culturing the seeded scaffold under conditions that promote one or more of proliferation, differentiation, and maturation of the seeded cells to form an engineered tissue having a thickness greater than 1 mm in at least one dimension.

2. The method of claim 1, wherein the scaffold comprises a plurality of hollow biomaterial tubes, each hollow biomaterial tube spaced apart to support efficient nutrient diffusion throughout the whole engineered tissue.

3. The method of claim 1, wherein the cells are cell spheroids and seeding comprises placing the cell spheroids between hollow biomaterial tubes of the scaffold.

4. The method of claim 1, wherein seeding comprises placing single cells adjacent to an outer surface of the one or more hollow biomaterial tubes.

5. The method of claim 4, wherein the outer surface of the one or more hollow biomaterial tubes comprises cell adhesion molecules.

6. The method of claim 1, wherein the hollow biomaterial tubes comprise a hydrogel.

7. The method of claim 6, wherein the hydrogel is degradable.

8. The method of claim 1, wherein the hollow biomaterial tubes comprise alginate.

9. The method of claim 8, wherein the alginate comprises alginate acid polymers, sodium alginate polymers, or modified alginate polymers, or combinations thereof.

10. The method of claim 8, wherein the alginate is dissolvable.

11. The method of claim 1, wherein the 3D scaffold is seeded with cells.

12. The method of claim 11, wherein the cells are selected from embryonic stem cells, induced pluripotent stem cells, cells differentiated from embryonic stem cells or induced pluripotent stem cells, cells reprogrammed from other cell types, primary cells, endothelial cells, umbilical vein endothelial cells, vascular smooth muscle cells, cancer cells, T cells, tissue stem cells, mammalian cells, plant cells, yeast, and bacterial cells, or a combination thereof.

13. The method of claim 1, further comprising seeding the hollow biomaterial tubes with cells.

14. The method of claim 13, wherein the cells comprise endothelial cells, vascular smooth muscle cells, or a combination thereof.

15. The method of claim 13, wherein the hollow biomaterial tubes are further seeded with growth factors.

16. The method of any of claims 13-15, wherein the engineered tissue comprises blood vessels.

17. An artificial tissue circulation system, the system comprising
(a) a directional fluid pumping device having a first inlet, a second inlet, and a reservoir; and
(b) a three-dimensional (3D) biocompatible scaffold comprising one or more hollow biomaterial tubes, each tube comprising a first end and a second end, wherein the first end is in fluid contact with the first inlet and the second end is in fluid contact with the second inlet, and wherein the first and second inlets are operable for introducing a fluid from the reservoir into the hollow biomaterial tube, thereby forming a fluid circuit between the directional fluid pumping device and the hollow biomaterial tube.

18. The system of claim 17, wherein the scaffold comprises a plurality of substantially parallel hollow biomaterial tubes.

19. The system of claim 17, wherein the hollow biomaterial tube comprises a hydrogel.

20. The system of claim 19, wherein the hydrogel comprises alginate.

21. The system of claim 18, wherein the alginate comprises alginate acid polymers, sodium alginate polymers, or modified alginate polymers, or combinations thereof.

22. The system of claim 17, wherein the 3D scaffold is seeded with cells.

23. The system of claim 22, wherein the cells are selected from embryonic stem cells, induced pluripotent stem cells, cells differentiated from embryonic stem cells or induced pluripotent stem cells, cells reprogrammed from other cell types, primary cells, endothelial cells, umbilical vein endothelial cells, vascular smooth muscle cells, cancer cells, T cells, tissue stem cells, mammalian cells, plant cells, yeast and bacterial cells, or a combination thereof.

24. The system of claim 17, wherein the reservoir comprises a cell culture medium.
